(19) United States
(12) Patent Application Publication
(10) Pub. No.: US 2008/0118441 A1
(43) Pub. Date: May 22, 2008

(54) COMPOSITIONS AND METHODS FOR THE TREATMENT OF GASTROINTESTINAL CONDITIONS

(76) Inventor: Joseph A. Washington, Shawnee, KS (US)

Correspondence Address: LATHROP & GAGE LC, 4845 PEARL EAST CIRCLE, SUITE 300, BOULDER, CO, 80301, US

(21) Appl. No.: 11/871,072

(22) Filed: Oct. 11, 2007

Related U.S. Application Data
(60) Provisional application No. 60/866,429, filed on Nov. 19, 2006.

(51) Int. Cl. A61K 36/185; A61K 31/201; A61K 36/68; A61P 1/04; A61K 9/12; A61K 31/732

(52) U.S. Cl. 424/43; 424/738; 424/769; 424/773; 514/54; 514/560

(57) ABSTRACT

Gastrointestinal conditions, such as gastroesophageal reflux disease, heartburn, acid indigestion, constipation, irritable bowel syndrome, stomach and intestinal ulcers, gastrointestinal tract inflammation, thrush, diverticulitis, ulcerative colitis, Crohn's disease, diarrhea and cancers of the esophagus, pancreas, stomach, intestines and colon, may be treated or prevented by administration of compositions containing a therapeutically effective amount of a fatty acid and a therapeutically effective amount of mucilaginous fiber. Fatty acids suitable for use in the compositions include omega-3 fatty acids, omega-6 fatty acids and/or omega-9 fatty acids.

### RELATED APPLICATIONS

This application claims the benefit of priority to commonly-owned and copending U.S. Provisional Patent Application No. 60/866,429, filed 19 Nov. 2006, which is incorporated herein by reference.

### BACKGROUND

Gastrointestinal conditions, such as gastroesophageal reflux disease (GERD), heartburn, acid indigestion, constipation, irritable bowel syndrome, stomach and intestinal ulcers, gastrointestinal tract inflammation, thrush, diverticulitis, ulcerative colitis, Crohn's disease, diarrhea and cancers of the esophagus, pancreas, stomach, intestines and colon, affect a large percentage of the population.

For example, GERD, or acid reflux, is a condition in which the liquid content of the stomach regurgitates into the esophagus. It is estimated that 25-40% of Americans have experienced symptomatic GERD at some point, while chronic GERD affects about 7-10% of Americans on a daily basis. Symptomatic GERD causes temporary discomfort in the form of heartburn, or acid indigestion, which manifests itself as a burning pain in the mid-chest, behind the breastbone, and in the mid-abdomen. When these symptoms persist in the form of chronic GERD they can have serious health consequences, such as inflammation and erosion of the esophagus lining (esophagitis).

Several over-the-counter and prescription medications are available to treat heartburn. For example, antacids such as TUMS® and Pepto-Bismol® contain salts that help buffer stomach acid, while pharmaceuticals such as Nexium® and Prilosec OTC® prevent gastric acid production. These products typically produce at least some unpleasant side effects and/or are unsuitable for use by persons taking contrainindicated medications for other medical conditions.

Therefore, a natural treatment for gastrointestinal conditions which produces few or no side effects and is suitable for use by persons having a wide variety of co-existent medical conditions would be useful.

### SUMMARY

The present instrumentalities advance the art and overcome the problems outlined above by providing compositions and methods for the treatment and prevention of gastrointestinal conditions.

In one embodiment, a composition for treatment or prevention of a gastrointestinal condition includes a therapeutically effective amount of a fatty acid and a therapeutically effective amount of mucilaginous fiber. The mucilaginous fiber is from a fiber source containing at least 30% soluble fiber, and the composition excludes glutamine.

In one embodiment, a composition for treatment or prevention of a gastrointestinal condition includes a therapeutically effective amount of a fatty acid including one or more of eicosapentaenoic acid and docosahexaenoic acid and a therapeutically effective amount of psyllium husk, where the composition excludes glutamine.

In one embodiment, a method for treating or preventing a gastrointestinal condition includes administering a composition including a therapeutically effective amount of a fatty acid and a therapeutically effective amount of mucilaginous fiber to a patient in need of such treatment. The mucilaginous fiber is from a fiber source containing at least 30% soluble fiber, and the composition excludes glutamine.

### DETAILED DESCRIPTION

There will now be shown and described compositions and methods for treating or preventing gastrointestinal conditions.

As used herein, the term “gastrointestinal condition” shall refer to a physiological condition that negatively impacts the gastrointestinal tract of a mammal, particularly a human. Gastrointestinal conditions that may be treated or prevented by the present compositions and methods include but are not limited to: gastroesophageal reflux disease (GERD), heartburn, acid indigestion, constipation, irritable bowel syndrome, stomach and intestinal ulcers, gastrointestinal tract inflammation, thrush, diverticulitis, ulcerative colitis, Crohn's disease, diarrhea and cancers of the esophagus, pancreas, stomach, intestines and colon.

In one embodiment, administration of a therapeutically effective amount of at least one fatty acid and a therapeutically effective amount of mucilaginous fiber significantly diminishes or eliminates symptoms associated with gastrointestinal conditions in mammals.

A “therapeutically effective amount” is intended to qualify the amount of active ingredient that will achieve the goal of fewer or less intense symptoms associated with a gastrointestinal condition. “Therapeutically effective” may also refer to improvement in disorder severity or the frequency of incidence over no treatment.

A “fatty acid” is a carboxylic acid that generally has a long unbranched aliphatic carbon chain. Fatty acids designated as omega-“x” fatty acids contain a double bond in the x^{th} carbon-carbon bond position, counting from the omega (methyl carbon) end of the chain, and may contain additional double bonds along the chain. Naturally occurring fatty acids may, for example, be isolated from fish and seed oils. Fatty acids suitable for use in the present compositions are typically C₁₈-C₂₄ fatty acids, and more typically C₂₀-C₂₂ fatty acids. Exemplary fatty acids include:

omega-3 fatty acids such as:
alpha-linolenic acid (CH₃(CH₂CH═CH)₃(CH₂)₇COOH),
stearidonic acid (CH₃(CH₂CH═CH)₄(CH₂)₇COOH),
eicosatetraeoic acid (CH₃(CH₂CH═CH)₄(CH₂)₆COOH),
eicosapentaenoic acid (CH₃(CH₂CH═CH)₅(CH₂)₃COOH),
docosapentanoic acid (CH₃(CH₂CH═CH)₅(CH₂)₅COOH) and
docosahexaenoic acid (CH₃(CH₂CH═CH)₆(CH₂)₂COOH),

omega-6 fatty acids such as:
linoleic acid (CH₃(CH₂)₄CH═CHCH₂CH═CH(CH₂)₇COOH),
gamma-linolenic acid (CH₃(CH₂)₄(CH═CHCH₂)₂CH═CH(CH₂)₄COOH),
eicosadienoic acid (CH₃(CH₂)₄(CH═CHCH₂)₂(CH₂)₈COOH),
dihomo-gamma-linolenic acid (CH₃(CH₂)₄(CH═CHCH₂)₃(CH₂)₅COOH),
arachidonic acid (CH₃(CH₂)₄(CH═CHCH₂)₄(CH₂)₂COOH),
docosadienoic acid (CH₃(CH₂)₄(CH═CHCH₂)₂(CH₂)₁₀COOH),
adrenic acid (CH₃(CH₂)₄(CH═CHCH₂)₄(CH₂)₄COOH),
docosapentaenoic acid (CH₃(CH₂)₄(CH═CHCH₂)₅CH₂COOH) and
calendic acid (CH₃(CH₂)₄(CH═CH)₃(CH₂)₆COOH),

and/or omega-9 fatty acids such as:
oleic acid (CH₃(CH₂)₇CH═CH(CH₂)₇COOH),
eicosenoic acid (CH₃(CH₂)₇CH═CH(CH₂)₉COOH),
mead acid (CH₃(CH₂)₇(CH═CHCH₂)₃(CH₂)₂COOH),
erucic acid (CH₃(CH₂)₇CH═CH(CH₂)₁₁COOH) and
nervonic acid (CH₃ (CH₂)₇CH═CH(CH₂)₁₃ COOH).

### In a preferred embodiment, the fatty acid is one or more of eicosapentaenoic acid and docosahexaenoic acid.

Administration may occur once daily, or be divided into several smaller doses over the course of 24 hours, e.g., half of the recommended dose may be administered twice daily, a third of the recommended dose may be administered every 8 hours, a quarter of the recommended dose may be administered every 6 hours, a sixth of the recommended dose may be administered every 4 hours, a twelfth of the recommended dose may be administered every 2 hours or a twenty-fourth of the recommended dose may be administered every hour. In a preferred embodiment, administration occurs once daily.

Compositions disclosed herein for daily administration, or administration over the course of 24 hours, include at least 1000 mg of one or more fatty acids. Ideally, a patient receives 1000 mg fatty acid at the start of treatment, and the dosage may be incrementally increased over the course of treatment until relief from the symptoms of one or more gastrointestinal conditions is achieved. Optimal dosages are expected to vary somewhat from patient to patient depending upon individual physiology and biochemistry. In one example, a patient may receive 1000 mg fatty acid per day. If symptoms are not alleviated by the third day, the fatty acid dosage may be increased, for example, to 1500 mg per day for the third and fourth days. If, by the fifth day, the symptoms are still not alleviated, the fatty acid dosage may be increased, for example, to 2000 mg per day for the fifth and sixth days. This incremental increase may continue every other day until a therapeutically effective amount of fatty acid is determined. A practical upper limit for fatty acid administration is about 8 grams per day. Thus, one or more fatty acids may be present in the disclosed compositions in a range between about 1 g and 8 g, preferably between about 2 g and 7 g, more preferably between about 2 g and 5 g, and most preferably between about 2.5 g and 3.5 g.

The disclosed compositions also contain at least one mucilaginous fiber, which is administered simultaneously with the one or more fatty acids mentioned above. “Mucilaginous fiber” is soluble fiber that is capable of absorbing a large amount of water to form a mucilage or gel. Preferably, the source of the mucilaginous fiber contains at least 30% soluble fiber, or at least 35%, 40%, 50%, 60%, 70%, 75% or 80% soluble fiber. Suitable fiber sources for use in the present compositions include, without limitation, pectin and pectin-like fibers, such as glucomannan; gums, such as locust bean gum, karaya gum, guar gum, gum arabic, carob bean gum, agar, alginates, gum acacia, caragum, carrageenan, inulin and xanthan gum; slippery elm bark; marshmallow root; and psyllium husk. In a prefelTed embodiment, the mucilaginous fiber is psyllium husk.

The mucilaginous fiber is typically present in a range of at least about 12 g (12 g=one tablespoon; 4 g=one teaspoon), preferably between about 4 g and 60 g, more preferably between about 8 g and 36 g, and most preferably between about 12 g and 24 g. The dosage of mucilaginous fiber may be incrementally increased (e.g., by about 4 g) every other day as discussed above for fatty acids.

Generally, fatty acids and mucilaginous fiber are available in powdered form, although highly concentrated fatty acids may be available as oils. These powders, or oils, may be used directly in gelatin capsules; mixed with binders and pressed into pills or suppositories; added to water or other consumable fluids, and optionally an emulsifying agent, to form a suspension; mixed with thickeners to form syrups; mixed with rubber to form chewing gum; mixed with liquefied sugars to form lozenges; aerosolized to form sprays; etc. As such, the disclosed compositions may be administered orally, e.g., as a pill, powder, suspension, syrup, lozenge, spray or gum; nasally, e.g., as an aerosol spray or mist; or rectally, e.g., as a suppository. The term “ingested” shall refer to administration by any of the aforementioned methods. It will be appreciated that the mode of administration may be determined by the gastrointestinal condition to be treated. For example, treatment of diverticulitis may be accomplished via suppository, while treatment of thrush may be accomplished via a lozenge and treatment of gastroesophageal reflux disease may be performed by oral administration of a suspension, e.g., where oil drops are suspended in liquid and/or where a solid is suspended in liquid.

Dosages disclosed herein refer to the amount of active ingredient in a composition. Other “additives”, components that are not active ingredients, may be included in the disclosed compositions, but additives do not contribute to the recommended weight or volume of active ingredient(s), e.g., fatty acid and/or mucilaginous fiber. Additives include, for example, pH adjusting agents, binders, emulsifiers, thickeners, aerosolizing agents, flavoring agents, preservatives, dyes and the like.

It has further been discovered that glutamine, a non-essential amino acid, counteracts the therapeutic effect of the disclosed compositions. In one example, the symptoms of a gastrointestinal condition will reappear rapidly if glutamine is ingested shortly after the present compositions. Exclusion of glutamine from the present compositions is therefore contemplated, and it may be advisable for persons being treated with the present compositions to reduce dietary consumption of glutamine, especially within one hour of ingesting the disclosed composition. Typically, an average person consumes 5-10 grams glutamine per day. In one embodiment, a person being treated with the present composition consumes no glutamine within one hour of ingestion, and preferably consumes less than about 5 grams glutamine per day, more preferably less than about 4.5 grams glutamine per day, and most preferably less than about 4 grams glutamine per day.

The reduced efficacy of the present compositions in the presence of glutamine is an important and surprising discovery in light of reports that suggest that intake of glutamine from food or supplements is essential when the body is affected by illness or injury. In particular, a significant body of evidence links glutamine-enriched diets with beneficial intestinal effects, such as aiding maintenance of gut barrier function, intestinal cell proliferation and differentiation, and reducing septic morbidity and the symptoms of irritable bowel syndrome.

pH Adjusting Agents

Compositions suitable for ingestion generally have a neutral pH, between about 6 and 9, preferably between about 6.5 and 8, more preferably between about 6.7 and 7.5. The pH of compositions disclosed herein may be adjusted by the addition of acidic, basic or buffering agents.

### Binders

Binders may be used with the disclosed compositions to congeal or otherwise hold powdered ingredients together. In one embodiment, gelatin capsules constitute a binder. In another embodiment, a binder may have cohesive properties. Typical cohesive binders include, without limitation, cornstarch; cellulose derivatives, such as methyl cellulose; gelatin and polyvinyl pyrrolidone (PVP). Binders for suppositories may include, for example, cocoa butter, polyethylene glycol and glycerin (glycerol and gelatin).

### Emulsifiers

Emulsifiers may be used in the present compositions to form suspensions of powdered ingredients. Suitable emulsifiers include biocompatible nonionic, anionic, cationic or amphoteric surfactants and biocompatible polymers, such as polyethylene glycol and salts thereof.

### Thickeners

Thickeners include starch and starch derivatives, such as hydroxyethyl starch or cross-linked starch; cellulosic materials such as hemicellulose (e.g., arabinoxylanes); cellulose and derivatives thereof (e.g., methyl cellulose and carboxymethyl cellulose); whey; gelatin and glycerol.

### Aerosolizing Agents

Aerosolizing agents, or propellants, may be used for example in nasal preparations. Suitable propellants include hydrofluoroalkanes, such as 1,1,1,2-tetrafluoroethane, 1,1,1,2,3,3,3-heptafluoro-n-propane, perfluoroethane, monochlorofluoromethane, 1,1-difluoroethane and combinations thereof. Devices and methods for delivering metered aerosolized doses are known in the art.

### Flavoring Agents

Synthetic and/or natural flavoring agents may be included in oral preparations. Suitable flavoring agents include, for example, sucrose, corn syrup, saccharin, aspartame, peppermint, spearmint, wintergreen, cinnamon, lemon, lime, orange, grape, cherry, apple, root bear, watermelon, chocolate, vanilla, strawberry and the like. In compositions intended for mammals other than humans, various other flavoring agents that appeal to the animal may be selected (e.g., meat, fish or vegetable flavoring agents).

### Preservatives

Preservatives may be added to the disclosed compositions. For example, ethylenediaminetetraacetic acid (EDTA) and its alkali salts act as chelating agents to bind metal ions that would otherwise facilitate metalloenzyme reactions that produce energy for bacterial cell replication. Other traditional preservatives include, for example, paraban, methyl paraban, ethyl paraban and alcohols.

### Dyes

One or more FD&C Certified (food grade) dyes may be included in the composition. Exemplary FD&C dyes include FD&C Red #40, FD&C Yellow #6, FD&C Yellow #5, FD&C Green #3, FD&C Blue #1, FD&C Orange #4 and combinations thereof.

### EXAMPLES

A correlational study involving six subjects that had either been diagnosed with GERD, or complained regularly of severe heartburn, was conducted. Compositions containing one or more fatty acids and psyllium husk were administered to each subject, and the subjects were asked to report any noticeable changes in the presence and/or severity of symptoms by way of independently created testimonials.

All six participants reported improvement of symptoms when the concentrations of active ingredients were adjusted to their specific physiology.

### Example 1

### Participant 1—Inventor

“Before taking the formula, I experienced painful heartburn symptoms daily. It was often difficult to get to sleep at night because of the pain. I tried several prescription drugs and either the relief didn't last for long or there were other side effects that made them not worth taking. I had given up on every over the counter and homeopathic medicine I had encountered up to that point.

After accidentally giving myself the combination of fiber and higher doses of Omega 3, my symptoms disappeared almost immediately. By the second day of taking the supplements, my symptoms were completely gone. The only thing left was a mild sensation of a bubble popping in my upper chest, esophagus region. However, that sensation was not painful at all and was much more preferable to the painful heartburn I'd experienced for years. Eventually that sensation disappeared as well and now I don't feel anything at all.

Since beginning the formula, I've intentionally skipped two or three days of dosing one or both of the supplements to make sure the relief is indeed a result of the combo. Each time, a milder form of the heartburn I had previously experienced would eventually return. I have been pain free for nearly two years now and it's clearly because of the formula.”

### Example 2

### Participant 2

“While participating in the acid reflux study, I noticed a significant reduction in heartburn. Prior to the study, I was suffering from heartburn on a daily basis. Since I was nursing there were few products that I could take other than Tums® which eased the heartburn but did not always get rid of it.

When I initially started the study, the product did not seem to work. After adjusting the formula, I noticed a significant reduction in heartburn. After a couple of weeks, I rarely suffered from heartburn. I did notice that I had better results when I ate a healthy diet.

Since the study, my heartburn has been significantly better. I rarely have problems with it. This is a dramatic improvement since I was taking the maximum number of Tums® that I could take nearly every day.”

### Example 3

### Participant 3

### As Conveyed to Experimenter

“I've had great success with the formula. My heartburn was pretty bad previous to trying it. Now I seldom feel that pain in my chest/esophagus or experience nausea like I did before. It only took a couple of days for me to begin feeling complete relief. I'm glad that I was able to participate in the study.”

### Example 4

### Participant 4

### As Conveyed to Experimenter

“Before the study, I often would experience painful sensations in my chest, especially after eating large meals or certain foods. Sometimes it would keep me up at nights.

Since trying the formula, my acid reflux/heartburn symptoms have been much, much better. I don't feel any of the same negative sensations. I'm even more regular. I go at the exact same time every day. A number of benefits have come from taking the formula.”

### Example 5

### Participant 5

“Before participating in the acid reflux study, I suffered from heartburn frequently. The heartburn symptoms regulated my life. I was not able to eat seasoned foods, without upsetting my heartburn. I started taking the formula on a regular basis and noticed changes in my symptoms. I adjusted the formula and my symptoms disappeared almost immediately. Once I was relieved of my symptoms I stopped taking the formula everyday. I now take the formula every other day and it works well for me. I did notice that I needed to take the formula consistently on a regular basis for my symptoms to be minimal.”

### Example 6

### Participant 6

“I have suffered from acid reflux for twenty years. I have been to many doctors and been prescribed every medication available. I have repeatedly tried different over the counter remedies. Nothing ever resolved the problem on a continuing basis.

I tried the formulation developed by Joe Washington and within one week I had absolutely no acid reflux symptoms. I have been using the formula for over 3 months and have yet to have the symptoms return.

I will continue to use this product as long as it is available. It has made a tremendous difference in my life with very little effort involved.”

Changes may be made in the above compositions and methods without departing from the scope hereof. It should thus be noted that the matter contained in the above description should be interpreted as illustrative and not in a limiting sense. The following claims are intended to cover all generic and specific features described herein, as well as all statements of the scope of the present compositions and methods, which, as a matter of language, might be said to fall there between.

What is claimed is:

1. A composition for treatment or prevention of a gastrointestinal condition comprising:
a therapeutically effective amount of a fatty acid; and
a therapeutically effective amount of mucilaginous fiber, the mucilaginous fiber from a fiber source containing at least 30% soluble fiber,
wherein the composition excludes glutamine.

2. The composition of claim 1, wherein the therapeutically effective amount of the fatty acid is at least 1000 mg.

3. The composition of claim 1, wherein the therapeutically effective amount of the mucilaginous fiber is at least 12 g.

4. The composition of claim 1, wherein the fatty acid is selected from the group consisting of omega-3 fatty acids, omega-6 fatty acids and omega-9 fatty acids.

5. The composition of claim 1, wherein the fatty acid is selected from the group consisting of alpha-linolenic acid, stearidonic acid, eicosatetraeoic acid, eicosapentaenoic acid, docosapentanoic acid, docosahexaenoic acid, linoleic acid, gamma-linolenic acid, eicosadienoic acid, dihomo-gamma-linolenic acid, arachidonic acid, docosadienoic acid, adrenic acid, docosapentaenoic acid, calendic acid, oleic acid, eicosenoic acid, mead acid, erucic acid and nervonic acid.

6. The composition of claim 5, wherein the fatty acid is selected from the group consisting of eicosapentaenoic acid and docosahexaenoic acid.

7. The composition of claim 1, wherein the fiber source is selected from the group consisting of pectin, gums, slippery elm bark, marshmallow root and psyllium husk.

8. The composition of claim 7, wherein the fiber source is psyllium husk.

9. A composition for treatment or prevention of a gastrointestinal condition comprising:
a therapeutically effective amount of a fatty acid including one or more of eicosapentaenoic acid and docosahexaenoic acid; and
a therapeutically effective amount of psyllium husk,
wherein the composition excludes glutamine.

10. The composition of claim 9, wherein the therapeutically effective amount of the fatty acid is at least 1000 mg.

11. The composition of claim 9, wherein the therapeutically effective amount of the psyllium husk is at least 12 g.

12. A method for treating or preventing a gastrointestinal condition comprising:
administering a composition including a therapeutically effective amount of a fatty acid and a therapeutically effective amount of mucilaginous fiber, the mucilaginous fiber from a fiber source containing at least 30% soluble fiber, to a patient in need of such treatment, wherein the composition excludes glutamine.

13. The method of claim 12, wherein the therapeutically effective amount of the fatty acid is at least 1000 mg.

14. The method of claim 12, wherein the therapeutically effective amount of the mucilaginous fiber is at least 12 g.

15. The method of claim 12, wherein the fatty acid is selected from the group consisting of omega-3 fatty acids, omega-6 fatty acids and omega-9 fatty acids.

16. The method of claim 12, wherein the fatty acid is selected from the group consisting of alpha-linolenic acid, stearidonic acid, eicosatetraeoic acid, eicosapentaenoic acid, docosapentanoic acid, docosahexaenoic acid, linoleic acid, gamma-linolenic acid, eicosadienoic acid, dihomo-gamma-linolenic acid, arachidonic acid, docosadienoic acid, adrenic acid, docosapentaenoic acid, calendic acid, oleic acid, eicosenoic acid, mead acid, erucic acid and nervonic acid.

17. The method of claim 12, wherein the fiber source is selected from the group consisting of pectin, gums, slippery elm bark, marshmallow root and psyllium husk.

18. The method of claim 12, wherein the gastrointestinal condition is selected from gastroesophageal reflux disease, heartburn, acid indigestion, constipation, irritable bowel syndrome, stomach and intestinal ulcers, gastrointestinal tract inflammation, thrush, diverticulitis, ulcerative colitis, Crohn's disease, diarrhea and cancers of the esophagus, pancreas, stomach, intestines and colon.

19. The method of claim 12, wherein the composition is administered orally, nasally or rectally.

20. The method of claim 12, wherein the composition is administered as one of a pill, a powder, a suspension, a syrup, a lozenge, an aerosol spray and a suppository.
